(19) United States
(12) Patent Application Publication
(10) Pub. No.: US 2022/0287776 A1
(43) Pub. Date: Sep. 15, 2022

(54) SYSTEMS AND METHODS FOR PERFORMANCE OF EXTERNAL BODY WALL DATA AND INTERNAL DEPTH DATA-BASED PERFORMANCE OF OPERATIONS ASSOCIATED WITH A COMPUTER-ASSISTED SURGICAL SYSTEM

(71) Applicant: Intuitive Surgical Operations, Inc., Sunnyvale, CA (US)

(72) Inventors: Robert G. Stricko, III, San Francisco, CA (US); Jacob L. Divone, Mountain View, CA (US); Glenn C. Stante, San Francisco, CA (US)

(21) Appl. No.: 17/634,024

(22) PCT Filed: Aug. 14, 2020
(86) PCT No.: PCT/US2020/046401

§ 371 Date: Feb. 9, 2022

Related U.S. Application Data
(60) Provisional application No. 62/888,236, filed on Aug. 16, 2019.

(51) Int. Cl. A61B 34/10; A61B 34/30; A61B 90/00; A61B 34/00

(52) U.S. Cl. CPC A61B 34/10; A61B 34/30; A61B 90/06; A61B 34/25; A61B 90/361; A61B 2034/105; A61B 2034/2059

(57) ABSTRACT

An exemplary operation management system is configured to obtain external body wall data representative of a three-dimensional model of an external body wall of a patient, obtain internal depth data representative of a depth map for an internal space of the patient, and perform, based on the external body wall data and the internal depth data, an operation associated with a computer-assisted surgical system configured to perform a procedure with respect to the patient.

### RELATED APPLICATIONS

The present application claims priority to U.S. Provisional Patent Application No. 62/888,236, filed on Aug. 16, 2019, and entitled “SYSTEMS AND METHODS FOR PERFORMANCE OF EXTERNAL BODY WALL DATA AND INTERNAL DEPTH DATA-BASED PERFORMANCE OF OPERATIONS ASSOCIATED WITH A COMPUTER-ASSISTED SURGICAL SYSTEM,” the contents of which are hereby incorporated by reference in their entirety.

### BACKGROUND INFORMATION

A computer-assisted surgical system is often used to perform minimally invasive and/or other types of surgical procedures within an internal space of a patient. For example, multiple surgical instruments may be coupled to manipulator arms of a computer-assisted surgical system, inserted into the patient by way of one or more ports (e.g., small orifices or incision sites) within an external body wall of the patient, and then robotically and/or teleoperatively controlled to perform a surgical procedure within the patient. Proper positioning of the one or more ports within the external body wall of the patient allows target anatomy within the patient to be adequately accessed with the one or more surgical instruments, minimizes the chance of collisions between the manipulator arms, and increases an effectiveness of the surgical procedure. However, proper port positioning depends on a number of factors that may be patient-specific. For example, proper port positioning often depends on a size and shape of a patient's external body wall, as well as a size, shape, and positioning of anatomical features within the internal space of the patient. Moreover, other operations associated with a computer-assisted surgical system, such as positioning of set-up joints to which the manipulator arms are connected, may depend on patient-specific characteristics.

### SUMMARY

The following description presents a simplified summary of one or more aspects of the systems and methods described herein. This summary is not an extensive overview of all contemplated aspects and is intended to neither identify key or critical elements of all aspects nor delineate the scope of any or all aspects. Its sole purpose is to present one or more aspects of the systems and methods described herein as a prelude to the detailed description that is presented below.

An exemplary system includes a memory storing instructions and a processor communicatively coupled to the memory and configured to execute the instructions to obtain external body wall data representative of a three-dimensional model of an external body wall of a patient, obtain internal depth data representative of a depth map for an internal space of the patient, and perform, based on the external body wall data and the internal depth data, an operation associated with a computer-assisted surgical system configured to perform a procedure with respect to the patient.

An exemplary method includes obtaining, by an operation management system, external body wall data representative of a three-dimensional model of an external body wall of a patient, obtaining, by the operation management system, internal depth data representative of a depth map for an internal space of the patient, and perform, by the operation management system based on the external body wall data and the internal depth data, an operation associated with a computer-assisted surgical system configured to perform a procedure with respect to the patient.

An exemplary non-transitory computer-readable medium stores instructions that, when executed, direct a processor of a computing device to obtain external body wall data representative of a three-dimensional model of an external body wall of a patient, obtain internal depth data representative of a depth map for an internal space of the patient, and perform, based on the external body wall data and the internal depth data, an operation associated with a computer-assisted surgical system configured to perform a procedure with respect to the patient.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary operation management system according to principles described herein.

FIG. 2 illustrates an exemplary configuration in which the system of FIG. 1 performs an operation associated with a computer-assisted surgical system based on external body wall data representative of a three-dimensional model of an external body wall of a patient and internal depth data representative of a depth map for an internal space of the patient according to principles described herein.

FIG. 3 illustrates an exemplary implementation in which the system of FIG. 1 obtains external body wall data and internal depth data from a depth sensor included in an imaging device according to principles described herein.

FIG. 4 illustrates an exemplary implementation in which a depth sensor is implemented by a time-of-flight sensor included in imaging device according to principles described herein.

FIG. 5 shows an exemplary implementation in which an illumination system is implemented by a single illumination source according to principles described herein.

FIG. 6 illustrates an exemplary implementation in which an illumination system is implemented by separate illumination sources according to principles described herein.

FIG. 7 illustrates an exemplary implementation in which an illumination source is integrated into a time-of-flight sensor according to principles described herein.

FIG. 8 illustrates an exemplary structural implementation of imaging device according to principles described herein.

FIG. 9 depicts a cross-sectional view of a shaft of an imaging device according to principles described herein.

FIG. 10 illustrates an exemplary implementation in which a depth sensor is implemented by visible light cameras included in imaging device according to principles described herein.

FIG. 11 shows an exemplary configuration in which the system of FIG. 1 obtains external body wall data from an external body wall data source according to principles described herein.

FIG. 12 shows an exemplary configuration in which an operation performed by the system of FIG. 1 is further based on kinematics data generated by computer-assisted surgical system according to principles described herein.

FIG. 13 shows an exemplary implementation of a computer-assisted surgical system according to principles described herein.

FIG. 14 is a simplified diagram showing an exemplary implementation of a manipulating system according to principles described herein.

FIG. 15 is a simplified diagram of a method of selecting a port location according to principles described herein.

FIGS. 16A-16B are simplified diagrams of different end effector positions and orientations within a workspace according to principles described herein.

FIG. 17 illustrates an exemplary method according to principles described herein.

FIG. 18 illustrates an exemplary computing device according to principles described herein.

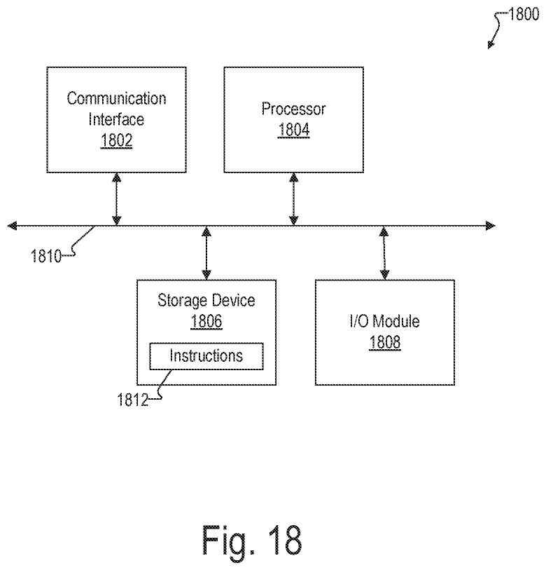

### DETAILED DESCRIPTION

Systems and methods for performance of external body wall data and internal depth data-based performance of operations associated with a computer-assisted surgical system are described herein. For example, an exemplary operation management system may obtain external body wall data representative of a three-dimensional model of an external body wall of a patient, obtain internal depth data representative of a depth map for an internal space of the patient, and perform, based on the external body wall data and the internal depth data, an operation associated with a computer-assisted surgical system configured to perform a procedure with respect to the patient.

The systems and methods described herein advantageously use external body wall data and internal depth data together to perform an operation associated with a computer-assisted surgical system. This may result in the operation being more precise, accurate, and effective than operations performed by or with respect to conventional computer-assisted surgical systems that do not have concurrent access to both types of data. These and other advantages and benefits of the systems and methods described herein will be made apparent here.

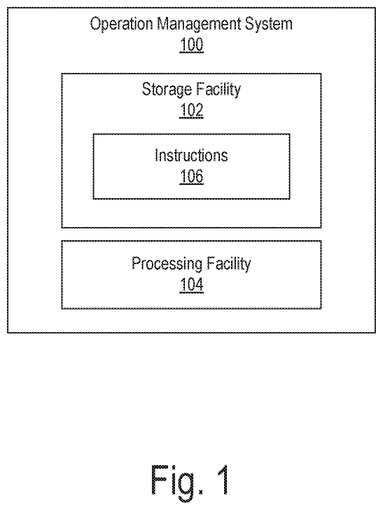

FIG. 1 illustrates an exemplary operation management system 100 (“system 100”) configured to perform external body wall data and internal depth data-based operations associated with a computer-assisted surgical system. As shown, system 100 may include, without limitation, a storage facility 102 and a processing facility 104 selectively and communicatively coupled to one another. Facilities 102 and 104 may each include or be implemented by hardware and/or software components (e.g., processors, memories, communication interfaces, instructions stored in memory for execution by the processors, etc.). For example, facilities 102 and/or 104 may be implemented by any component in the computer-assisted surgical system itself. As another example, facilities 102 and/or 104 may be implemented by a computing device separate from and communicatively coupled to the computer-assisted surgical system. In some examples, facilities 102 and 104 may be distributed between multiple devices and/or multiple locations as may serve a particular implementation.

Storage facility 102 may maintain (e.g., store) executable data used by processing facility 104 to perform one or more of the operations described herein. For example, storage facility 102 may store instructions 106 that may be executed by processing facility 104 to perform one or more of the operations described herein. Instructions 106 may be implemented by any suitable application, software, code, and/or other executable data instance. Storage facility 102 may also maintain any data received, generated, managed, used, and/or transmitted by processing facility 104.

Processing facility 104 may be configured to perform (e.g., execute instructions 106 stored in storage facility 102 to perform) various operations described herein. For example, processing facility 104 may be configured to obtain external body wall data representative of a three-dimensional model of an external body wall of a patient, obtain internal depth data representative of a depth map for an internal space of the patient, and perform, based on the external body wall data and the internal depth data, an operation associated with a computer-assisted surgical system configured to perform a procedure with respect to the patient. These and other operations that may be performed by system 100 (e.g., processing facility 104) are described herein.

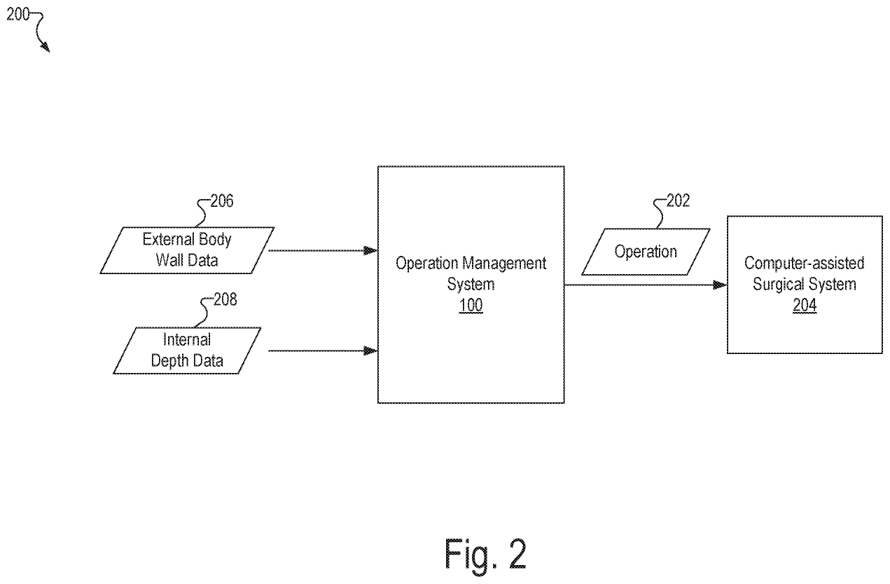

FIG. 2 illustrates an exemplary configuration in which system 100 performs an operation 202 associated with a computer-assisted surgical system 204 based on external body wall data 206 representative of a three-dimensional model of an external body wall of a patient and internal depth data 208 representative of a depth map for an internal space of the patient. System 100 may obtain external body wall data 206 and internal depth data 208 in any suitable manner, examples of which are provided herein.

Computer-assisted surgical system 204 may be implemented by any suitable surgical system that uses robotic and/or teleoperation technology to perform a procedure (e.g., a minimally invasive surgical procedure) with respect to a patient. Exemplary computer-assisted surgical systems are described herein.

Operation 202 may include any suitable operation performed with respect to computer-assisted surgical system 204. In cases where system 100 is implemented by computer-assisted surgical system 204 itself, operation 202 may be performed by computer-assisted surgical system 204. Examples of operation 202 are described herein.

Various exemplary manners in which system 100 may obtain external body wall data 206 and internal depth data 208 will now be described.

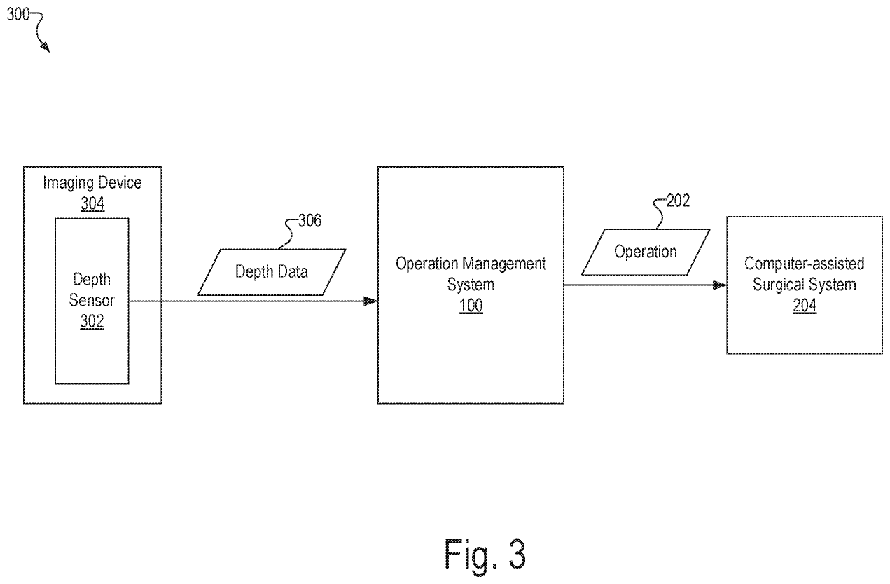

FIG. 3 illustrates an exemplary implementation 300 in which system 100 obtains external body wall data 206 and internal depth data 208 from a depth sensor 302 included in an imaging device 304. As shown, depth sensor 302 is configured to generate depth data 306 representative of a depth map of a scene imaged by imaging device 304. As described herein, depending on a positioning of imaging device 304 with respect to a patient, depth data 306 may be representative of either external body wall data 206 or internal depth data 208.

Imaging device 304 may be implemented by an endoscope or other camera device configured to capture images of a scene. In some examples, imaging device 304 may be configured to be attached to and controlled by computer-assisted surgical system 204. In alternative examples, imaging device 304 may be hand-held and operated manually by an operator (e.g., a surgeon).

In some examples, the scene captured by imaging device 304 may include a surgical area associated with a patient. The surgical area may, in certain examples, be entirely disposed within the patient and may include an area within the patient at or near where a surgical procedure is planned to be performed, is being performed, or has been performed. For example, for a minimally invasive surgical procedure being performed on tissue internal to a patient, the surgical area may include the tissue, anatomy underlying the tissue, as well as space around the tissue where, for example, surgical instruments used to perform the surgical procedure are located. In certain example implementations, the surgical area entirely disposed within the patient may be referred to as an “internal space.” As described herein, any internal anatomy of the patient (e.g., vessels, organs, and/or tissue) and/or surgical instruments located in the internal space may be referred to as objects and/or structures.

The surgical area included in the scene captured by imaging device 304 may, in some examples, also include an area external to the patient. For example, imaging device 304 may be used to image an external body wall of the patient.

Depth sensor 302 included in imaging device 304 may be implemented by any suitable sensor configured to generate depth data 306. For example, as described herein, depth sensor 302 may be implemented by a time-of-flight sensor, stereoscopic cameras, and/or any other suitable components as may serve a particular implementation. Depending on the positioning of imaging device 304, depth data 306 may be representative of a depth map for an external body wall of the patient or a depth map for an internal space of the patient.

In implementation 300, system 100 is configured to obtain external body wall data 206 by directing depth sensor 302 to scan (e.g., image) an external body wall of the patient while imaging device 304 is external to the patient. In this configuration, depth data 306 generated by depth sensor 302 is representative of a depth map for the external body wall and may be accordingly referred to herein as “external depth data.”

Depth sensor 302 may scan the external body wall of the patient in any suitable manner. In some examples, imaging device 304 is coupled to computer-assisted surgical system 204 (e.g., attached to a manipulator arm of computer-assisted surgical system 204) while depth sensor 302 scans the external body wall. Alternatively, imaging device 304 may be manually held by a user (e.g., a surgeon) while depth sensor 302 scans the external body wall. In some examples, the scanning may be performed while the patient is insufflated.

System 100 may receive depth data 306 acquired by depth sensor 302 while imaging device 304 is external to the patient in any suitable manner. For example, system 100 may direct depth sensor 302 to transmit depth data 306 to system 100. System 100 may then use the depth data 306 as external body wall data 206.

In implementation 300, imaging device 304 and depth sensor 302 are also used by system 300 to obtain internal depth data 208. For example, depth sensor 302 may be aimed at an internal space of the patient through a camera port formed through the external body wall of the patient. This may be performed, for example, by inserting imaging device 304 through the camera port such that a distal end of imaging device 304 is within the internal space of the patient. In this configuration, system 100 may direct depth sensor 302 to scan the internal space to acquire depth data 306. In this configuration, depth data 306 is representative of a depth map for the internal space of the patient and may be accordingly referred to herein as “internal depth data.”

System 100 may receive depth data 306 acquired by depth sensor 302 while imaging device 304 is aimed at the internal space of the patient in any suitable manner. For example, system 100 may direct depth sensor 302 to transmit depth data 306 to system 100. System 100 may then use the depth data 306 as internal depth data 208.

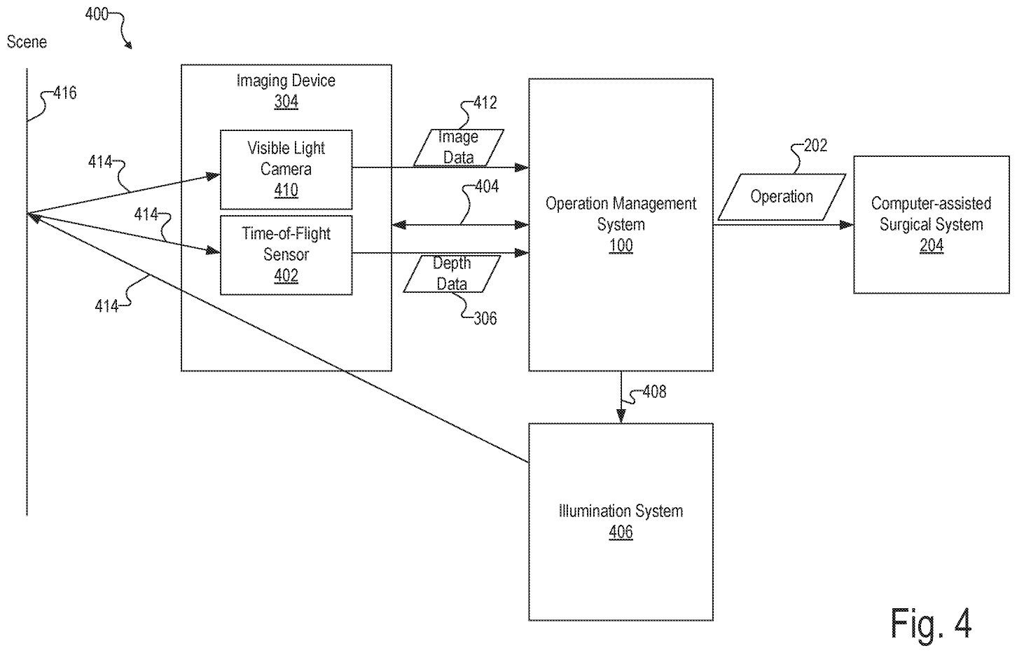

FIG. 4 illustrates an exemplary implementation 400 in which depth sensor 302 is implemented by a time-of-flight sensor 402 included in imaging device 304. While time-of-flight sensor 402 is shown in FIG. 4 and referred to in the examples provided herein, any other type of depth sensor separate from (i.e., physically distinct from) a visible light camera also included in imaging device 304 may additionally or alternatively be used to implement depth sensor 302. For example, depth sensor 302 may alternatively be implemented by a structured light sensor, an interferometer, and/or any other suitable sensor configured to acquire depth data as may serve a particular implementation.

In implementation 400, system 100 may obtain depth data 306 by directing time-of-flight sensor 402 to acquire depth data 306 and receiving depth data 306 from time-of-flight sensor 402. For example, system 100 may direct time-of-flight sensor 402 to acquire external depth data representative of a depth map for an external body wall of a patient by scanning the external body wall while imaging device 304 is external to the patient. System 100 may receive the external depth data from time-of-flight sensor 402 and use the external depth data as external body wall data 206. System 100 may also direct time-of-flight sensor 402 to acquire internal depth data 208 while time-of-flight sensor 402 is aimed at the internal space of the patient through a camera port formed through the external body wall of the patient.

To this end, in implementation 400, system 100 is communicatively coupled to imaging device 304 by way of a bidirectional communication link 404 and to an illumination system 406 by way of a communication link 408. Communication links 404 and 408 may each be implemented by any suitable wired and/or wireless communication medium as may serve a particular implementation. System 100 may use communication links 404 and 408 to direct time-of-flight sensor 402 to acquire depth data 306 and receive depth data 306 from time-of-flight sensor 402, as described herein.

As shown, imaging device 304 includes time-of-flight sensor 402 and a visible light camera 410 (“camera 410”), which is configured to generate image data 412 representative of a two-dimensional visible light image of a scene. Time-of-flight sensor 402 may be implemented by one or more photodetectors (e.g., one or more single photon avalanche diode (“SPAD”) detectors), CCD sensors, CMOS sensors, and/or any other suitable configuration configured to obtain depth data of a scene. Camera 410 may be implemented by any suitable image sensor, such as a charge coupled device (“CCD”) image sensor, a complementary metal-oxide semiconductor (“CMOS”) image sensor, or the like.

In some examples, system 100 may be configured to control an operation of imaging device 304 (e.g., by controlling an operation of camera 410 and time-of-flight sensor 402). For example, system 100 may include one or more camera control units (“CCUs”) configured to control various parameters (e.g., activation times, auto exposure, etc.) of camera 410 and/or time-of-flight sensor 402.

System 100 may additionally or alternatively be configured to provide operating power for components included in imaging device 304. For example, while imaging device 304 is communicatively coupled to system 100, system 100 may transmit operating power to camera 410 and time-of-flight sensor 402 in the form of one or more power signals.

System 100 may be configured to use imaging device 304 and illumination system 406 to acquire depth data 306 and image data 412. In some examples, depth data 306 and image data 412 may be used to generate stereoscopic images of a scene. This will be described in more detail below.

Illumination system 406 may be configured to emit light 414 (e.g., at the direction of system 100) used to illuminate a scene to be imaged by imaging device 304. The light 414 emitted by illumination system 406 may include visible light and/or non-visible light (e.g., infrared light). As shown, light 414 may travel to the scene through imaging device 304 (e.g., by way of an illumination channel within imaging device 304 that may be implemented by one or more optical fibers, light guides, lenses, etc.). Various implementations and configurations of illumination system 406 are described herein.

As shown, light 414 emitted by illumination system 406 may reflect off a surface 416 within a scene being imaged by imaging device 304. In cases where imaging device 304 is external to the patient, surface 416 represents a surface of the external body wall of the patient. In cases where imaging device 304 is aimed at an internal space of the patient, surface 416 represents a surface within the internal space (e.g., a surface of an organ and/or other tissue).

Visible light camera 410 and time-of-flight sensor 402 may each detect the reflected light 414. Visible light camera 410 may be configured to generate, based on the detected light, image data 412 representative of a two-dimensional visible light image of the scene including surface 416. Time-of-flight sensor 402 may be configured to generate, based on the detected light, depth data 306. Image data 412 and depth data 306 may each have any suitable format.

To generate a stereoscopic image of a scene, system 100 may direct illumination system 406 to emit light 414. System 100 may also activate (e.g., turn on) visible light camera 410 and time-of-flight sensor 402. Light 414 travels to the scene and reflects off of surface 416 (and, in some examples, one or more other surfaces in the scene). Camera 410 and time-of-flight sensor 402 both detect the reflected light 414.

Camera 410 (and/or other circuitry included in imaging device 304) may generate, based on detected light 414, image data 412 representative of a two-dimensional visible light image of the scene. This may be performed in any suitable manner. Visible light camera 410 (and/or other circuitry included imaging device 304) may transmit image data 412 to system 100. This may also be performed in any suitable manner.

Time-of-flight sensor 402 may generate, based on detected light 414, depth data 306 representative of a depth map of the scene (e.g., a depth map of surface 416). This may be performed in any suitable manner. For example, time-of-flight sensor 402 may measure an amount of time that it takes for a photon of light 414 to travel from illumination system 406 to time-of-flight sensor 402. Based on this amount of time, time-of-flight sensor 402 may determine a depth of surface 416 relative to a position of time-of-flight sensor 402. Data representative of this depth may be represented in depth data 306 in any suitable manner. For example, the depth map represented by depth data 306 may include an array of depth values (e.g., Z-buffer values) corresponding to each pixel in an image.

Time-of-flight sensor 402 (and/or other circuitry included imaging device 304) may transmit depth data 306 to system 100. This may be performed in any suitable manner.

System 100 may receive image data 412 and depth data 306 and perform one or more processing operations on image data 412 and depth data 306. For example, based on image data 412 and depth data 306, system 100 may generate a right-side perspective image of the scene and a left-side perspective image representative of the scene. This may be performed in any suitable manner. System 100 may then direct display devices to concurrently display the right and left-side perspective images in a manner that forms a stereoscopic image of the scene. In some examples, the display devices are included in and/or communicatively coupled to computer-assisted surgical system 204.

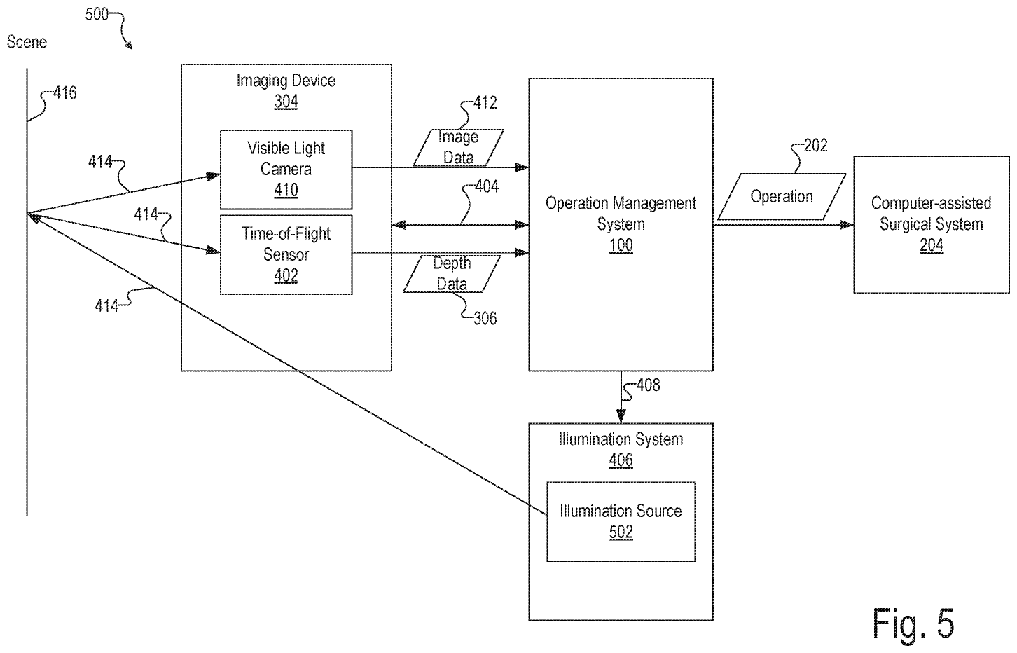

FIG. 5 shows an exemplary implementation 500 in which illumination system 406 is implemented by a single illumination source 502. Illumination source 502 may be configured to emit visible light 414-1.

Visible light 414-1 may include one or more color components. For example, visible light 414-1 may include white light that includes a full spectrum of color components (e.g., red, green, and blue color components). The red color component has wavelengths between approximately 945 and 800 nanometers (“nm”). The green color component has wavelengths between approximately 820 and 860 nm. The blue color component has wavelengths between approximately 750 and 790 nm.

In some examples, visible light 414-1 is biased to include more of one color component than another color component. For example, visible light 414-1 may be blue-biased by including more of the blue color component than the red and green color components.

In implementation 500, time-of-flight sensor 402 is configured to also detect visible light 414-1. Accordingly, the same illumination source 502 may be used for both camera 410 and time-of-flight sensor 402.

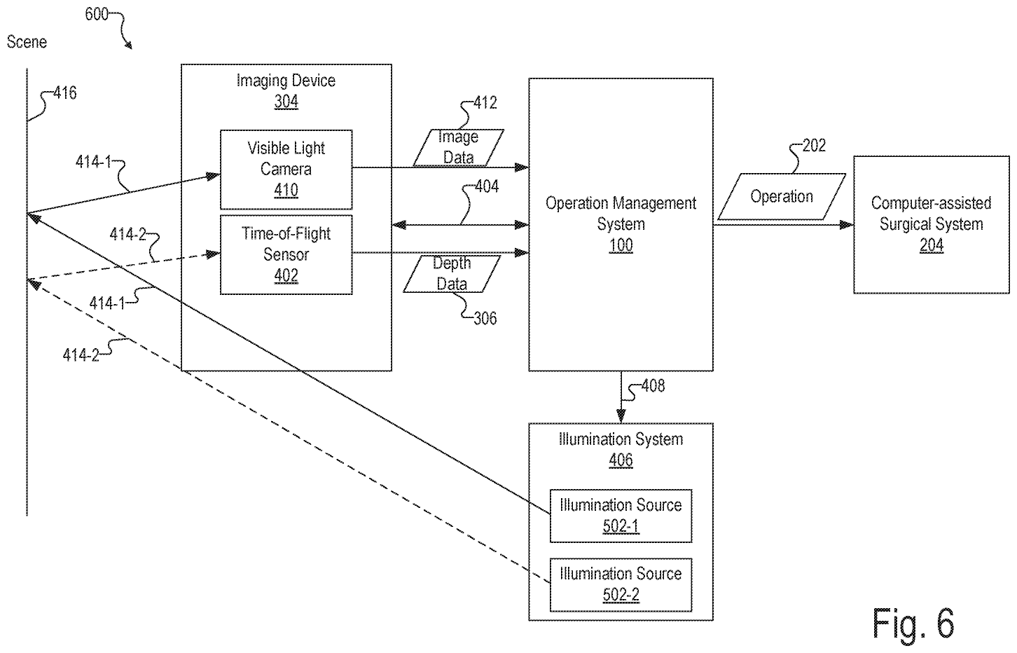

FIG. 6 illustrates an exemplary implementation 600 in which illumination system 406 is implemented by separate illumination sources 502-1 and 402-2. In implementation 600, illumination source 502-1 is configured to emit visible light 414-1 that is detected by camera 410. Illumination source 502-2 is configured to emit light 414-2 that reflects from surface 416 and is detected by time-of-flight sensor 402. In some examples, light 414-2 is non-visible light, such as infrared light. By having separate illumination sources 502 for camera 410 and time-of-flight sensor 402, camera 410 and time-of-flight sensor 402 may be configured to operate independently.

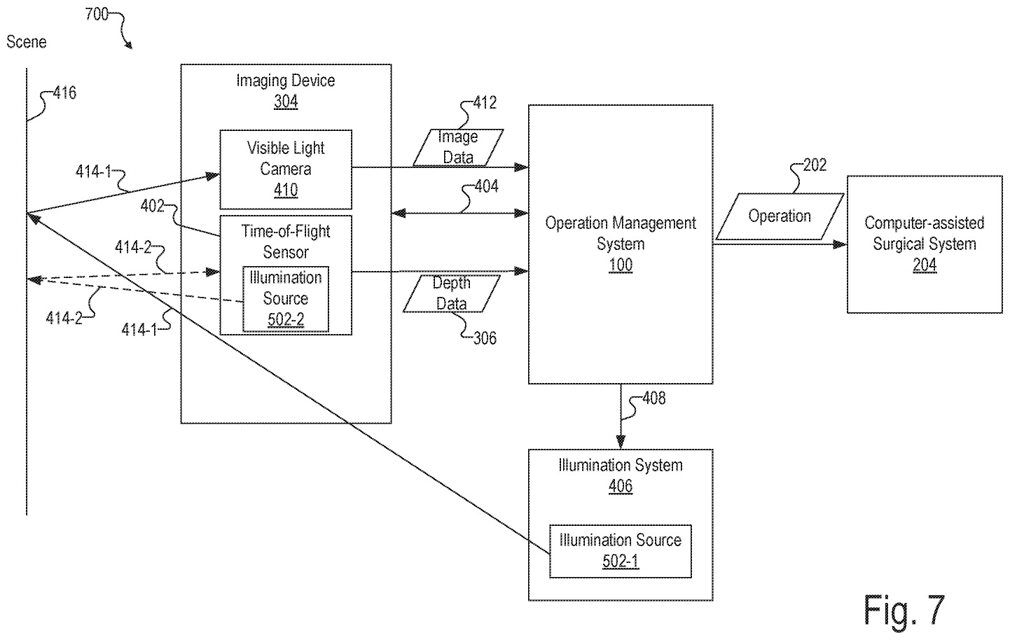

FIG. 7 illustrates an exemplary implementation 700 in which illumination source 502-2 is integrated into time-of-flight sensor 402. In implementation 700, system 100 may control (e.g., activate) illumination source 502-2 by transmitting instructions to time-of-flight sensor 402.

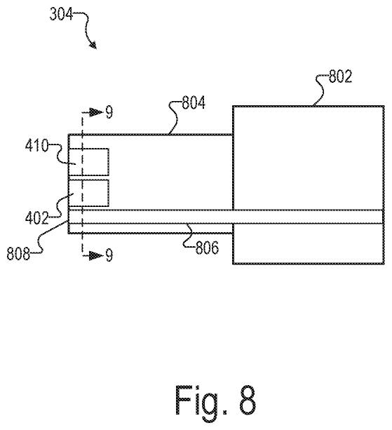

FIG. 8 illustrates an exemplary structural implementation of imaging device 304. As shown, imaging device 304 includes a camera head 802 and a shaft 804 coupled to and extending away from camera head 802. Camera head 802 and shaft 804 together implement a housing of imaging device 304. Imaging device 304 may be manually handled and controlled (e.g., by a surgeon performing a surgical procedure on a patient). Alternatively, camera head 802 may be coupled to a manipulator arm of computer-assisted surgical system 204. In this configuration, imaging device 304 may be controlled by computer-assisted surgical system 204 using robotic and/or teleoperation technology.

As shown, an illumination channel 806 may pass through camera head 802 and shaft 804. Illumination channel 806 is configured to provide a conduit for light emitted by illumination system 406 to travel to a scene that is being imaged by imaging device 304.

A distal end 808 of shaft 804 may be positioned at or near a scene that is to be imaged by imaging device 304. For example, distal end 808 of shaft 804 may be inserted into a patient. In this configuration, imaging device 304 may be used to capture images of anatomy and/or other objects within the patient.

Camera 410 and time-of-flight sensor 402 may be located anywhere along shaft 804 of imaging device 304. In the example shown in FIG. 8, camera 410 and time-of-flight sensor 402 are located at distal end 808 of shaft 804. This configuration may be referred to as a “chip on tip” configuration. Alternatively, camera 410 and/or time-of-flight sensor 402 may be located more towards camera head 802 and/or within camera head 802. In these alternative configurations, optics (e.g., lenses, optical fibers, etc.) included in shaft 804 and/or camera head 206 may convey light from a scene to camera 410 and/or time-of-flight sensor 402.

In some examples, camera 410 and time-of-flight sensor 402 may be staggered at different distances from distal end 808 of shaft 804. By staggering the distances of camera 410 and time-of-flight sensor 402 from distal end 808 of shaft 804, imaging device 304 may take on a tapered configuration with a reduced size (e.g., diameter) towards distal end 808 of the shaft 804, which may be helpful for inserting the imaging device 304 into an internal space of a patient.

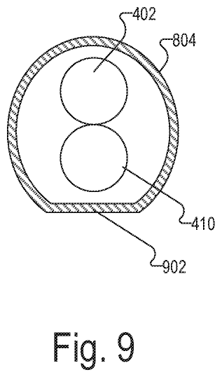

FIG. 9 depicts a cross-sectional view of shaft 804 of imaging device 304 taken along lines 9-9 in FIG. 8. As shown, shaft 804 includes a relatively flat bottom surface 902. With reference to this bottom surface 902, time-of-flight sensor 402 is positioned above camera 410. Such positioning may allow for a more narrow shaft 804 compared to shafts of conventional imaging devices that have two cameras side-by-side in order to acquire stereoscopic images. It will be recognized that camera 410 and time-of-flight sensor 402 may have any suitable relative position within shaft 804 as may serve a particular implementation.

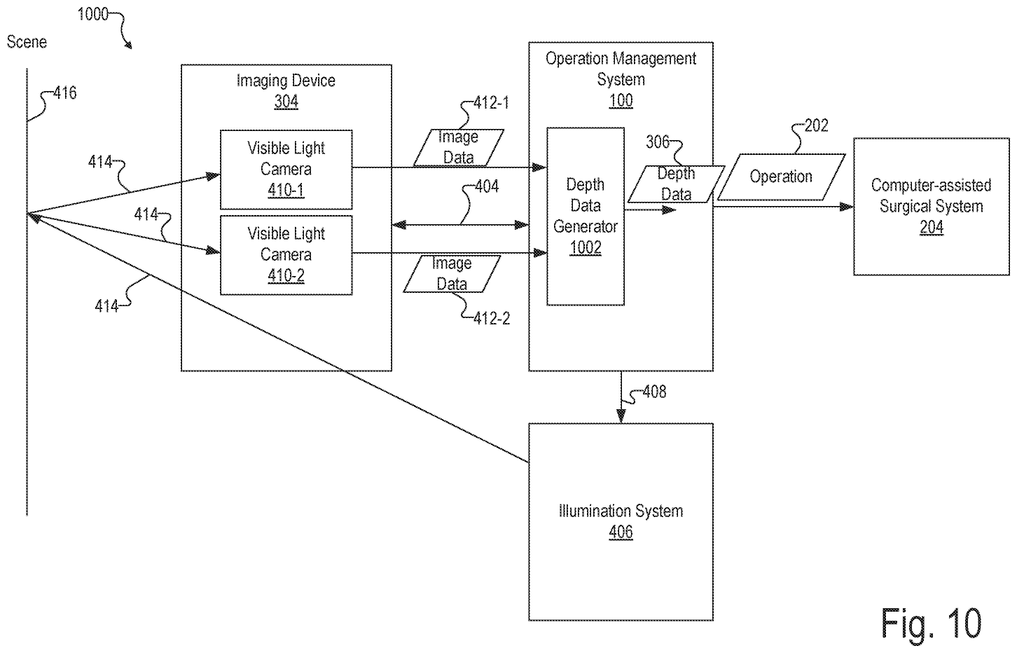

FIG. 10 illustrates an exemplary implementation 1000 in which depth sensor 402 is implemented by visible light cameras 410-1 and 410-2 included in imaging device 304. In implementation 1000, system 100 may obtain depth data 306 by directing camera 410-1 to acquire a first image (e.g., a first two-dimensional image) of an internal space of a patient, directing camera 410-2 to acquire a second image (e.g., a second two-dimensional image) of the internal space of the patient, and generating, based on the first and second images, the depth map represented by depth data 306.

In FIG. 10, the first image acquired by camera 410-1 is represented by image data 412-1 and the second image acquired by camera 410-2 is represented by image data 412-2. As shown, image data 412-1 and 412-2 are transmitted to a depth data generator 1002 implemented by system 100. Depth data generator 1002 may use any visible image-based technique to determine depth data 306 based on image data 412-1 and 412-2.

Other configurations of imaging device 304 are possible in accordance with the systems and methods described herein. For example, imaging device 304 may include multiple cameras 410 and/or multiple time-of-flight sensors 402. To illustrate, imaging device 304 may include two cameras 410 in combination with a single time-of-flight sensor 402. In these embodiments, depth data may be generated based on the images acquired by both cameras 410. Depth data generated by time-of-flight sensor 402 may be used to fine tune or otherwise enhance the depth data generated based on the images acquired by both cameras 410.

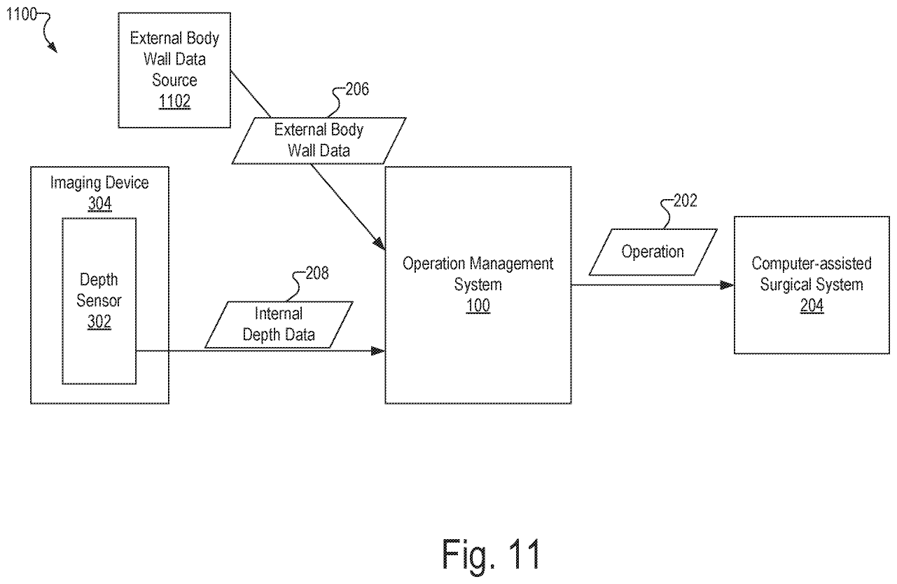

In some examples, system 100 may obtain external body wall data 206 from a source other than imaging device 204. For example, FIG. 11 shows an exemplary configuration 1100 in which system 100 obtains external body wall data 206 from an external body wall data source 1102 (“source 1102”) that is different than imaging device 304. Source 1102 may be implemented by a computer-aided tomography (CT) scanner, a magnetic resonance imaging (MRI) device, an ultrasound device, a three-dimensional scanning (LIDAR) device, and/or any other suitable alternative imaging device. As another example, source 1102 may be implemented by a computing device configured to maintain previously acquired external body wall data 206. For example, external body wall data 206 may be generated for a patient during a first surgical procedure. External body wall data 206 may be stored by a computing device and used for the patient during a second surgical procedure subsequent to the first surgical procedure.

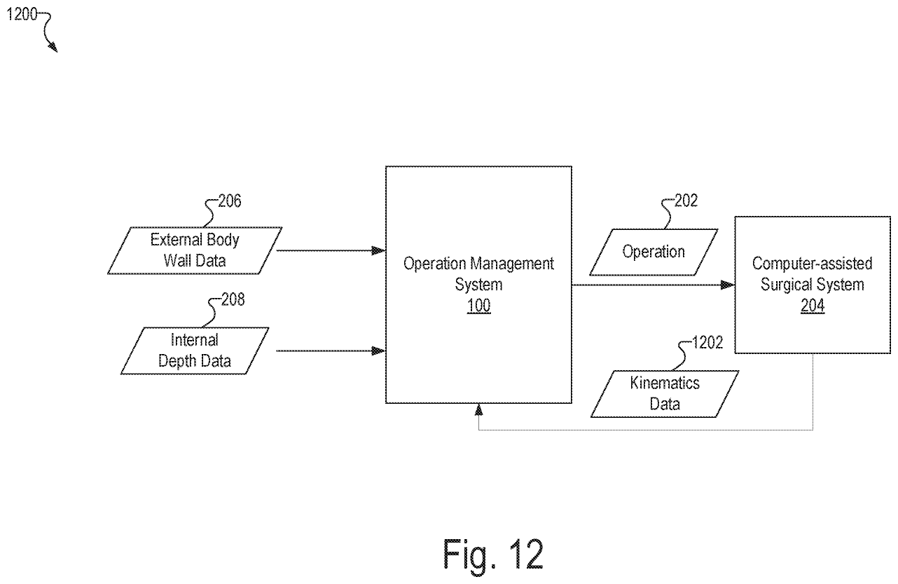

FIG. 12 shows an exemplary configuration 1200 in which operation 202 performed by system 100 is further based on kinematics data 1202 generated by computer-assisted surgical system 204. Hence, in configuration 1200, operation 202 is based on external body wall data 206, internal depth data 208, and kinematics data 1202. Exemplary operations 202 that are based on external body wall data 206, internal depth data 208, and kinematics data 1202 are described herein.

Kinematics data 1202 may be representative of any type of kinematics information associated with one or more components of computer-assisted surgical system 204 (e.g., one or more manipulator arms and/or set-up joints of computer-assisted surgical system 204). Kinematics data 1202 may additionally or alternatively be representative of any type of kinematics information associated with one or more components coupled to computer-assisted surgical system 204 (e.g., imaging device 304 and/or one or more surgical instruments). Such kinematics information may include, but is not limited to, information indicating displacement, orientation, position, and/or movement of one or more components of computer-assisted surgical system 204 and/or one or more components coupled to computer-assisted surgical system 204. For example, kinematics data 1202 for imaging device 304 generated while imaging device 304 is coupled to computer-assisted surgical system 204 may indicate a positioning and/or orientation of imaging device 304 when depth data 306 and/or image data 412 is acquired by imaging device 304. Such positioning and/or orientation may be with respect to a particular reference position and/or orientation as may serve a particular implementation. For example, kinematics data 1202 may indicate that imaging device 304 is a certain distance away from the external body wall of a patient when external depth data is acquired by depth sensor 302, or that a distal end of imaging device 304 is inserted a certain distance into the patient when internal depth data 208 is acquired by depth sensor 302.

Kinematics data 1202 may be generated by computer-assisted surgical system 204 in any suitable manner. For example, one or more transducers and/or sensors within computer-assisted surgical system 204 may track displacement, orientation, position, movement, and/or other types of kinematic information and output kinematics data 1202 (or sensor output data used by computer-assisted surgical system 204 to generate kinematics data 1202).

In some examples, system 100 may use kinematics data 1202 to register external body wall data 206 with internal depth data 208. For example, imaging device 304 may be attached to a manipulator arm of computer-assisted surgical system 204 while depth sensor 302 (e.g., time-of-flight sensor 402) scans the external body wall of a patient and generates depth data 306 used as external body wall data 206. The imaging device 304 may then be inserted into the internal space of the patient to generate depth data 306 used as internal depth data 208. During both of these operations, computer-assisted surgical system 204 may track a position of imaging device 304 and output kinematics data 1202 representative of the position. Kinematics data 1202 may then be used by system 100 to register external body wall data 206 with internal depth data 208.

As used herein, registration of external body wall data 206 with internal depth data 208 refers to mapping external body wall data 206 with internal depth data 208 in a manner that generates a combined three-dimensional model (also referred to herein as a “patient model”) of the external body wall of the patient and the internal space of the patient. In this manner, system 100 may know where certain internal structures are located with respect to different positions on the external body wall of the patient. Hence, the performance of operation 202 by system 100 may be based on the registration of external body wall data 206 with internal depth data 208.

Various examples of operation 202 that may be performed by system 100 with respect to computer-assisted surgical system 204 based on external body wall data 206 and internal depth data 208 will now be provided. These examples are merely illustrative of the many different types of operations that may be performed by system 100 based on external body wall data 206 and internal depth data 208 in accordance with the systems and methods described herein.

In some examples, system 100 may perform operation 202 by identifying, based on external body wall data 206 and internal depth data 208, a port location on an external body wall of a patient through which computer-assisted surgical system 204 is to insert a surgical instrument into an internal space of the patient.

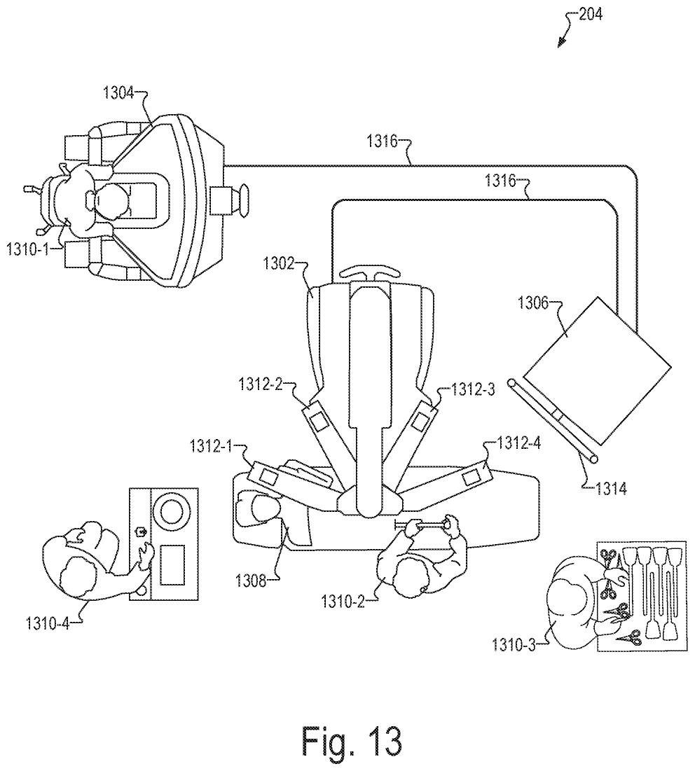

To illustrate, FIG. 13 shows an exemplary implementation of computer-assisted surgical system 204. It will be recognized that the components shown in FIG. 13 are merely exemplary, and that additional or alternative components may be included in computer-assisted surgical system 204 as may serve a particular implementation.

As shown, computer-assisted surgical system 204 includes a manipulating system 1302, a user control system 1304, and an auxiliary system 1306 communicatively coupled one to another. Computer-assisted surgical system 204 may be utilized by a surgical team to perform a computer-assisted surgical procedure on a patient 1308. As shown, the surgical team may include a surgeon 1310-1, an assistant 1310-2, a nurse 1310-3, and an anesthesiologist 1310-4, all of whom may be collectively referred to as “surgical team members 1310.” Additional or alternative surgical team members may be present during a surgical session as may serve a particular implementation.

While FIG. 13 illustrates an ongoing minimally invasive surgical procedure, it will be understood that computer-assisted surgical system 204 may similarly be used to perform open surgical procedures or other types of surgical procedures that may similarly benefit from the accuracy and convenience of computer-assisted surgical system 204. Additionally, it will be understood that the surgical session throughout which computer-assisted surgical system 204 may be employed may not only include an operative phase of a surgical procedure, as is illustrated in FIG. 13, but may also include preoperative, postoperative, and/or other suitable phases of the surgical procedure. A surgical procedure may include any procedure in which manual and/or instrumental techniques are used on a patient to investigate or treat a physical condition of the patient.

As shown in FIG. 13, manipulating system 1302 may include a plurality of manipulator arms 1312 (e.g., manipulator arms 1312-1 through 1312-4) to which a plurality of surgical instruments may be coupled. Each surgical instrument may be implemented by any suitable surgical tool (e.g., a tool having tissue-interaction functions), medical tool, imaging device (e.g., an endoscope), sensing instrument (e.g., a force-sensing surgical instrument), diagnostic instrument, or the like that may be used for a computer-assisted surgical procedure on patient 1308 (e.g., by being at least partially inserted into patient 1308 and manipulated to perform a computer-assisted surgical procedure on patient 1308). While manipulating system 1302 is depicted and described herein as including four manipulator arms 1312, it will be recognized that manipulating system 1302 may include only a single manipulator arm 1312 or any other number of manipulator arms as may serve a particular implementation.

Manipulator arms 1312 and/or surgical instruments attached to manipulator arms 1312 may include one or more displacement transducers, orientational sensors, and/or positional sensors used to generate raw (i.e., uncorrected) kinematics information. One or more components of computer-assisted surgical system 204 may be configured to use the kinematics information to track (e.g., determine positions of) and/or control the surgical instruments.

User control system 1304 may be configured to facilitate control by surgeon 1310-1 of manipulator arms 1312 and surgical instruments attached to manipulator arms 1312. For example, surgeon 1310-1 may interact with user control system 1304 to remotely move or manipulate manipulator arms 1312 and the surgical instruments. To this end, user control system 1304 may provide surgeon 1310-1 with imagery (e.g., high-definition 3D imagery) of a surgical area associated with patient 1308 as captured by an imaging system (e.g., any of the medical imaging systems described herein). In certain examples, user control system 1304 may include a stereo viewer having two displays where stereoscopic images of a surgical area associated with patient 1308 and generated by a stereoscopic imaging system may be viewed by surgeon 1310-1. Surgeon 1310-1 may utilize the imagery to perform one or more procedures with one or more surgical instruments attached to manipulator arms 1312.

To facilitate control of surgical instruments, user control system 1304 may include a set of master controls. These master controls may be manipulated by surgeon 1310-1 to control movement of surgical instruments (e.g., by utilizing robotic and/or teleoperation technology). The master controls may be configured to detect a wide variety of hand, wrist, and finger movements by surgeon 1310-1. In this manner, surgeon 1310-1 may intuitively perform a procedure using one or more surgical instruments.

Auxiliary system 1306 may include one or more computing devices configured to perform primary processing operations of computer-assisted surgical system 204. In such configurations, the one or more computing devices included in auxiliary system 1306 may control and/or coordinate operations performed by various other components (e.g., manipulating system 1302 and user control system 1304) of computer-assisted surgical system 204. For example, a computing device included in user control system 1304 may transmit instructions to manipulating system 1302 by way of the one or more computing devices included in auxiliary system 1306. As another example, auxiliary system 1306 may receive, from manipulating system 1302, and process image data representative of imagery captured by an imaging device attached to one of manipulator arms 1312.

In some examples, auxiliary system 1306 may be configured to present visual content to surgical team members 1310 who may not have access to the images provided to surgeon 1310-1 at user control system 1304. To this end, auxiliary system 1306 may include a display monitor 1314 configured to display one or more user interfaces, such as images (e.g., 2D images) of the surgical area, information associated with patient 1308 and/or the surgical procedure, and/or any other visual content as may serve a particular implementation. For example, display monitor 1314 may display images of the surgical area together with additional content (e.g., graphical content, contextual information, etc.) concurrently displayed with the images. In some embodiments, display monitor 1314 is implemented by a touchscreen display with which surgical team members 1310 may interact (e.g., by way of touch gestures) to provide user input to computer-assisted surgical system 204.

Manipulating system 1302, user control system 1304, and auxiliary system 1306 may be communicatively coupled one to another in any suitable manner. For example, as shown in FIG. 13, manipulating system 1302, user control system 1304, and auxiliary system 1306 may be communicatively coupled by way of control lines 1316, which may represent any wired or wireless communication link as may serve a particular implementation. To this end, manipulating system 1302, user control system 1304, and auxiliary system 1306 may each include one or more wired or wireless communication interfaces, such as one or more local area network interfaces, Wi-Fi network interfaces, cellular interfaces, etc.

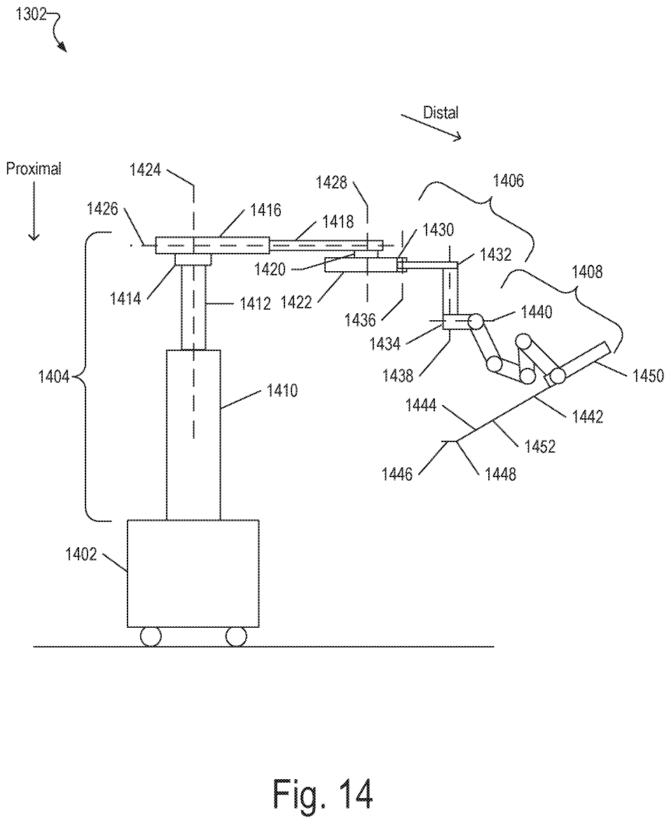

FIG. 14 is a simplified diagram showing an exemplary implementation of manipulating system 1302. As shown in FIG. 14, manipulating system 1302 may include a mobile cart 1402, which enables manipulating system 1302 to be transported from location to location, such as between operating rooms or within an operating room to better position manipulating system 1302 near a patient table. In alternative embodiments, manipulating system 1302 includes a stationary base.

Starting at the proximal end with mobile cart 1402 is a set-up structure 1404. Coupled to a distal end of the set-up structure are a series of set-up joints 1406. Coupled to a distal end of the set-up joints 1406 is a manipulator 1408, such as a universal surgical manipulator. In some examples, the series of set-up joints 1406 and manipulator 1408 may implement one of the manipulator arms 1312. Although manipulating system 1302 is shown with only one series of set-up joints 1406 and a corresponding manipulator 1408, it will be recognized that manipulating system 1302 may include more than one series of set-up joints 1406 and corresponding manipulators 1408 so that the manipulating system 1302 is equipped with multiple manipulator arms.

As shown in FIG. 14, set-up structure 1404 includes a two part column including column links 1410 and 1412. Coupled to an upper or distal end of column link 1412 is a shoulder joint 1414. Coupled to shoulder joint 1414 is a two-part boom including boom links 1416 and 1418. At the distal end of boom link 1418 is a wrist joint 1420, and coupled to wrist joint 1420 is an orientation platform 1422.

The links and joints of set-up structure 1404 include various degrees of freedom for changing a position and orientation (i.e., the pose) of the orientation platform 1422. For example, the two-part column may be used to adjust a height of the orientation platform 1422 by moving the shoulder joint 1414 up and down along an axis 1426. The orientation platform 1422 may additionally be rotated about the mobile cart 1402, the two-part column, and the axis 1426 using the shoulder joint 1414. The horizontal position of the orientation platform 1422 may also be adjusted along an axis 1426 using the two-part boom. The orientation of the orientation platform 1422 may also adjusted by rotation about an axis 1428 using the wrist joint 1420. Thus, subject to the motion limits of the links and joints in the set-up structure 1404, the position of the orientation platform 1422 may be adjusted vertically above the mobile cart 1402 using the two-part column. The positions of the orientation platform 1422 may also be adjusted radially and angularly about the mobile cart 1402 using the two-part boom and the shoulder joint 1414, respectively. And the angular orientation of the orientation platform 1422 may also be changed using the wrist joint 1420.

The orientation platform 1422 may be used as a mounting point for one or more manipulator arms. The ability to adjust the height, horizontal position, and orientation of the orientation platform 1422 about the mobile cart 1402 provides a flexible set-up structure for positioning and orienting the one or more manipulator arms about a workspace, such as a patient, located near the mobile cart 1402. FIG. 14 shows a single manipulator arm coupled to the orientation platform using a first set-up joint 1430. Although only one manipulator arm is shown, it will be recognized that multiple manipulator arms may be coupled to the orientation platform 1422 using additional first set-up joints.

The first set-up joint 1430 forms the most proximal portion of the set-up joints 1406 section of the manipulator arm. The set-up joints 1406 may further include a series of joints and links. As shown in FIG. 14, the set-up joints 1406 include at least links 1432 and 1434 coupled via one or more joints (not expressly shown). The joints and links of the set-up joints 1406 include the ability to rotate the set-up joints 1406 relative to the orientation platform 1422 about an axis 1436 using the first set-up joint 1430, adjust a height of the link 1434 relative to the orientation platform along an axis 1438, and rotate the manipulator at least about an axis 1440 at the distal end of the link 1434. The set-up joints 1406 may further include additional joints, links, and axes permitting additional degrees of freedom for altering a position and/or orientation of the manipulator 1408 relative to the orientation platform 1422.

The manipulator 1408 is coupled to the distal end of the set-up joints 1406 and includes additional links and joints that permit control over a position and orientation of a surgical instrument 1442 mounted at a distal end of the manipulator 1408. Surgical instrument 1442 includes an elongate shaft 1444 that is coupled between manipulator 1408 and an end effector 1446 via an optional articulated wrist 1448. The degrees of freedom in the manipulator 1408 may permit at least control of the roll, pitch, and yaw of the elongate shaft 1444 relative to the distal end of the set-up joints 1406. In some examples, the degrees of freedom in the manipulator 1408 may further include the ability to advance and/or retreat elongate shaft 1444 along an insertion carriage or spar 1450 so as to move end effector 1446 nearer to or farther away from manipulator 1408 along a longitudinal axis of surgical instrument 1442. Additional control over the orientation of end effector 1446 relative to manipulator 1408 may be controlled using optional wrist 1448. In some examples, the degrees of freedom of the set-up joints 1406 and the manipulator 1408 may further be controlled so as to maintain a remote center 1452 about a point on the surgical instrument 1442. In some examples, the remote center 1452 may correspond to a port in a patient so that as the surgical instrument 1442 is used, the remote center 1452 remains stationary to limit stresses on the anatomy of the patient at the remote center 1452. In some examples, the surgical instrument 1442 may be an imaging device such as an endoscope, a gripper, a surgical tool such as a cautery or a scalpel, and/or the like.

Controlling the location where surgical instrument 1442 is inserted into an internal space of a patient, such as by inserting elongate shaft 1444 through a cannula located at a port for accessing the interior anatomy of the patient, is desirable for the flexible operation of manipulating system 1302 and surgical instrument 1442. In some examples, if the location of the port is too close to target tissue, surgical instrument 1442 and end effector 1446 may not have sufficient range of motion to access, interact with, and manipulate the target tissue. If the location of the port is too far from the target tissue, end effector 1446 may not be able to reach the target tissue. If the location of the port is poorly chosen, there may be intervening tissues between the port and the target tissue which elongate shaft 1444 and end effector 1446 may not be able to maneuver around and/or elongate shaft 1444 and end effector 1446 may not have a comfortable or practical approach orientation to the target tissue. When manipulating system 1302 includes multiple manipulators 1408 and multiple instruments 1442, the placement of their corresponding ports too close together may result in a higher likelihood of interference and/or collisions between manipulator arms (e.g., corresponding spars 1450 and/or manipulators 1408), instruments 1442, and/or other portions of manipulating system 1302.

Conventional approaches to selecting port locations have typically relied on general port placement rules determined empirically from previous use of manipulating system 1302 and, common sense based on a basic understanding of a workspace configuration, such as the typical anatomy of a patient for a surgical procedure. As an example, in the case of an upper abdominal surgery, recommendations for the port locations may include placing the port for an imaging device (e.g., an endoscope) at the umbilicus and locating additional ports along a diagonal line perpendicular to target anatomy and through the umbilicus along with a recommended spacing. Additional recommendations may include locating one or more of the ports above (superior to) or below (inferior to) the diagonal line to accommodate instruments 1442 with different kinds of end effectors 1446. While these types of guidelines may provide a good location for the ports, the guidelines do not always have sufficient flexibility to address variations in a workspace (e.g., patients with larger or smaller anatomy and/or patients with unusual anatomy due to previous procedures, the presence of lesions, and/or the like), variations in instruments and/or procedures, variations in operator preferences, and/or the like.

Hence, with reference to FIGS. 13-14, system 100 may use external body wall data 206 and internal depth data 208 to identify a port location on an external body wall of patient 1308 through which computer-assisted surgical system 204 is to insert a surgical instrument (e.g., surgical instrument 1442) into an internal space of patient 1308.

For example, system 100 may identify, based on external body wall data 206 and internal depth data 208, a port location that allows surgical instrument 1442 to access, through the port location, a structure within the internal space of the patient while avoiding collision with an additional surgical instrument 1442. As another example, system 100 may identify a port location that allows surgical instrument 1442 to access a structure within the internal space without a manipulator arm (e.g., manipulator arm 1312-1) to which surgical instrument 1442 is attached colliding with a different manipulator arm (e.g., manipulator arm 1312-2). These operations may be performed in any suitable manner. For example, based on external body wall data 206 and internal depth data 208, system 100 may ascertain a depth of the structure and its relative position with respect to various locations on the external body wall of patient 1308. Based on this, system 100 may select an appropriate port location on the external body wall that allows access to the structure while preventing (or at least minimizing a chance for) a collision between surgical instruments 1442 and/or between manipulator arms 1312 (e.g., collisions between spars 1450).

As another example, system 100 may identify a port location that allows surgical instrument 1442 and the manipulator arm 1312 to which surgical instrument 1442 is attached to avoid unintentional contact with patient 1308. For example, based on external body wall data 206 and internal depth data 208, system 100 may determine a positioning of manipulator arm 1312 that avoids contact with the external body wall and/or any other external feature (e.g., a face) of the patient. This positioning may be used to determine the port location.

In some examples, one or more additional types of data may be used together with external body wall data 206 and internal depth data 208 to identify the port location. For example, system 100 may determine, for a candidate port location, at least one of a reachability metric indicating an ability of the surgical instrument to reach a target structure located in the internal space of the patient using a candidate port location, an anthropomorphic metric indicating an ease with which a user may manipulate the surgical instrument introduced into the internal space of the patient through the candidate port location, a collision volume for portions of the computer-assisted surgical system proximal to the candidate port location, the collision volume corresponding to a volume swept by the portions of the computer-assisted surgical system proximal to the candidate port location, and a collision metric indicating a likelihood of a collision between portions of the computer-assisted surgical system proximal to the candidate port location. System 100 may use one or more of these metrics together with external body wall data 206 and internal depth data 208 to identify the port location (e.g., by designating the candidate port location as the port location).

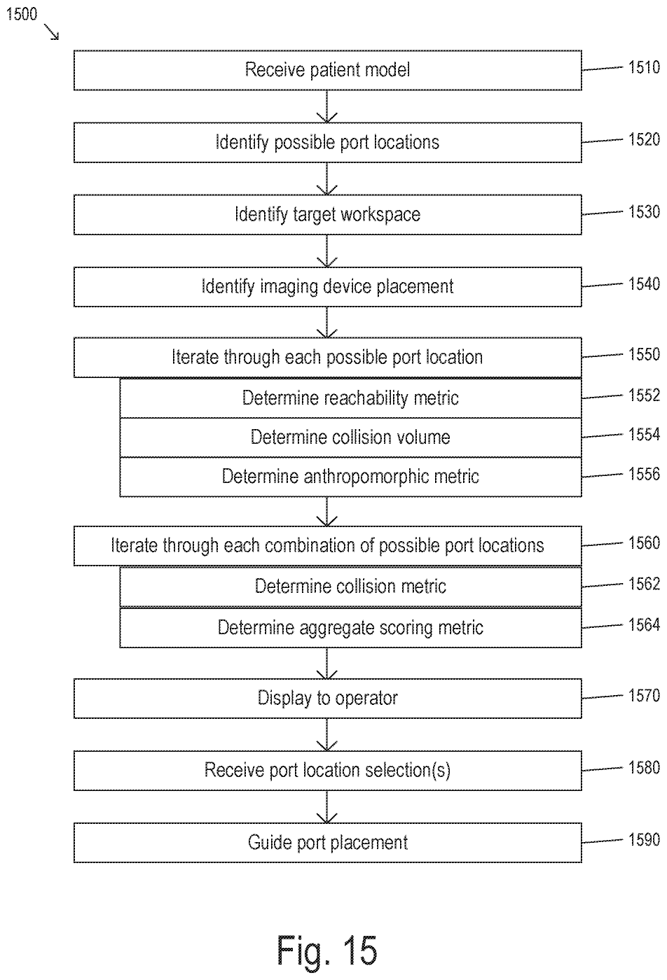

To illustrate, FIG. 15 is a simplified diagram of a method 1500 of selecting port locations according to some embodiments. Method 1500 may be used together with the external body wall data and internal depth data-based methods described herein to select a port location on the external body wall of a patient through which a computer-assisted surgical system is to insert a surgical instrument into the internal space of the patient. One or more of the operations 1510-1590 of method 1500 may be performed by system 100. Embodiments related to method 1500 are described more fully in PCT Publication No. WO2019089226A2, the contents of which are incorporated herein by reference in their entirety.

In some embodiments, method 1500 may be used to identify port locations, evaluate each of the port locations, evaluate combinations of port locations, aid an operator in selecting and utilizing suitable port locations, and/or the like. In some examples, method 1500 may be used to evaluate the port locations for one or more surgical instruments, such as surgical instrument 1442, being teleoperated using a manipulating system, such as manipulating system 1302. The operations shown in FIG. 15 are illustrative only. Method 1500 may include additional or alternative operations as may serve a particular implementation.

At operation 1510, a patient model is received. The patient model may be generated based on external body wall data 206 and internal depth data 208, as described herein.

At operation 1520, an initial set of possible port locations (also referred to herein as “candidate port locations”) are identified. In some examples, knowledge about the target tissue for the procedure (e.g., a location of a lesion to be biopsied or resected) is mapped to the patient model data obtained during operation 1510 and a plurality of possible port locations are identified on the external body wall of the patient. In some examples, the possible port locations are limited to those portions of the external body wall that are within a threshold distance of the target anatomy so as to limit the possible port locations to those that are reachable using available surgical instruments. In some examples, the possible port locations may be limited based on general knowledge of anatomy, such as restricting port locations for an upper abdominal procedure to those located on an anterior portion of the patient anatomy below the rib cage and above the waist line. Each of the possible port locations may correspond to locations of existing orifices in the exterior anatomy of the patient and/or potential incision sites.

At operation 1530, a target workspace (e.g., an internal space of the patient) is identified. In some examples, the location of the target tissue and the procedures to be performed on the target tissue are used to identify a procedure site envelope or workspace around the target tissue where one or more surgical instruments are to be manipulated so as to access, grasp, manipulate, and/or otherwise interact with the target tissue. As an example, an end effector for grasping, stapling, and cutting may use a target workspace that includes room to approach the target tissue, articulate jaws into a desired orientation, move the jaws around the target tissue, perform the grasping, stapling, and cutting of the target tissue, and then retreat from the target tissue. In some examples, this target workspace may be determined using kinematic models of the corresponding surgical instrument and end effector and identifying a swept volume through which the surgical instrument and/or end effector moves to perform the procedure.

At operation 1540, an imaging device placement is identified. In some examples, the location of the imaging device may be set to a default location determined based on the procedure to be performed (e.g., using a port located at the umbilicus for an upper abdominal procedure), operator preference, operator direction, and/or the like. In some examples, in addition to identification of the placement of the imaging device, additional information associated with the imaging device may be obtained including one or more of a model of the imaging device, a direction of view of the imaging device, a field of view of the imaging device (e.g., a range of angles relative to a direction of view access that may be captured using the imaging device, an aspect ratio of images captured by the imaging device, an actual or perceived working distance between the imaging device and the target anatomy and/or target workspace, and/or the like).

At operation 1550, each of the possible port locations identified during operation 1520 is iterated through to evaluate its suitability as a port location. As each of the possible port locations is considered, the analyses of operations 1552-1556 are repeated to determine metrics usable to characterize corresponding aspects of each of the port locations as to level of suitability for use with the contemplated procedure.

At operation 1552, a reachability metric is determined for a port location, The reachability metric is a kinematic measure of how well the target tissue and/or the target workspace identified during operation 1530 may be reached using a surgical instrument inserted into the workspace via the port location. In some embodiments, the reachability metric may address the ability of the surgical instrument to reach the target tissue from the port location. In some examples, the reachability metric may be determined by determining an articulation volume (also called a reachable swept volume) within the patient anatomy that is reachable by an end effector (e.g., end effector 1446) by articulating an elongate shaft (e.g., elongate shaft 1444) of a surgical instrument (e.g., surgical instrument 1442) through a roughly conical space with an apex at the port location (e.g., remote center 1452) as the pitch, yaw, and level of insertion are varied. In some examples, when the surgical instrument includes an articulated wrist (e.g., articulated wrist 1448), the reachable swept volume may additionally include points reachable by articulating the articulated wrist as the pitch, yaw, and level of insertion of the surgical instrument are also adjusted. In some examples, the pitch and/or yaw may be limited by range of motion limits of the surgical instrument or the manipulator to which the surgical instrument is mounted and/or the insertion depth may be limited by a length of the elongate shaft and/or the relative location of the remote center relative to the manipulator. In some examples, additional factors that may further limit the reachable swept volume include the capabilities of the manipulating system, a current position and/or orientation of one or more joints of the manipulating system, a model of the manipulating system, an orientation of the patient, an orientation of an operating table on which the patient is placed, a location of the manipulating system relative to the patient, and/or the like. In some examples, one or more kinematic models of the surgical instrument and/or the manipulator to which the surgical instrument is mounted may be used to determine the reachable swept volume.

In some embodiments, the reachability metric may address the ability of the surgical instrument to reach and maneuver around the target tissue from the port location and may be characterized as an ability to reach a dexterous workspace related to the target workspace identified during operation 1520. In some examples, a dexterous swept volume similar to the reachable swept volume described above may be determined with points in the dexterous swept volume being additionally limited to those points in the workspace that may be reached subject to the ability of the points to be reached over a range of articulations in the articulated wrist. In some examples, one or more kinematic models of the surgical instrument and/or the manipulator to which the surgical instrument is mounted may be used to determine the dexterously reachable swept volume.

In some examples, the reachability metric may be a binary pass-fail metric indicating whether the target tissue is reachable and/or dexterously reachable using the surgical instrument from the port location. In some examples, the reachability metric may an analog value, such as in the range between 0 and 1 inclusive, indicating a relative quality of the reachability and/or the dexterous reachability. In some examples, the analog value may be assigned based on how much of the target tissue is reachable by the surgical instrument from the port location (e.g., how much of the target tissue is within the reachable swept volume). In some examples, the analog value may be assigned based on how much of the insertion range of the surgical instrument is used to reach the target tissue with 0 representing not reachable and 1 representing that the surgical instrument may reach the target tissue from the port location using a predetermined percentage of the full insertion. In some examples, the analog value may be determined based on how far the target tissue is from half the full insertion of the surgical instrument according to Equation 1, where the full insertion is length L and the distance between the port location and the target tissue is d. In some examples, other equations may be used.

Length Analog Reachability Metric=1−|d−0.5L|/0.5L Equation 1

In some examples, the analog value may be determined based on how far the target tissue is from the center line of the swept volume so that when the target tissue is closer to the center line of the swept volume, the higher the corresponding reachability metric. In some examples, the analog value may be determined according to Equation 2, where a is the angle between the center line of the swept volume and the line between the port location and the target tissue and A is the largest pitch and/or yaw angle of the surgical instrument. In some examples, other equations may be used that favor target tissue locations closer to the center line.

Angle Analog Reachability Metric=a/A Equation 2

In some examples, the length and angle analog reachability metrics may both be used with their values being combined using any triangular norm function, such as minimum, multiplication, and/or the like.

At operation 1554, a collision volume is determined. In order to manipulate the surgical instrument within the workspace, one or more portions of the surgical instrument and/or the manipulator to which the surgical instrument is mounted that are proximal to the port location are also subject to motion that results in the one or more portions of the surgical instrument and/or the manipulator to which the surgical instrument is mounted moving through a swept volume (also referred to as a collision volume or region of activity) external to the patient and/or the workspace. When more than one surgical instrument and corresponding manipulator and/or repositionable arm are used, overlaps between their respective collision volumes indicate a potential for collisions during a procedure. In some examples, the collision volume for the port location may be determined by using one or more kinematic models of the surgical instrument, the manipulator to which the surgical instrument is mounted, and/or the repositionable arm to which the manipulator is mounted and noting that collision volume as the surgical instrument is manipulated through its complete range of motion through the port location. In some examples, the portions of the surgical instrument, manipulator, and/or repositionable arm used to generate the collision volume may be a subset of the joints and linkages of surgical instrument, manipulator, and/or repositionable arm, such as only spar 1450 in the examples of FIG. 14.

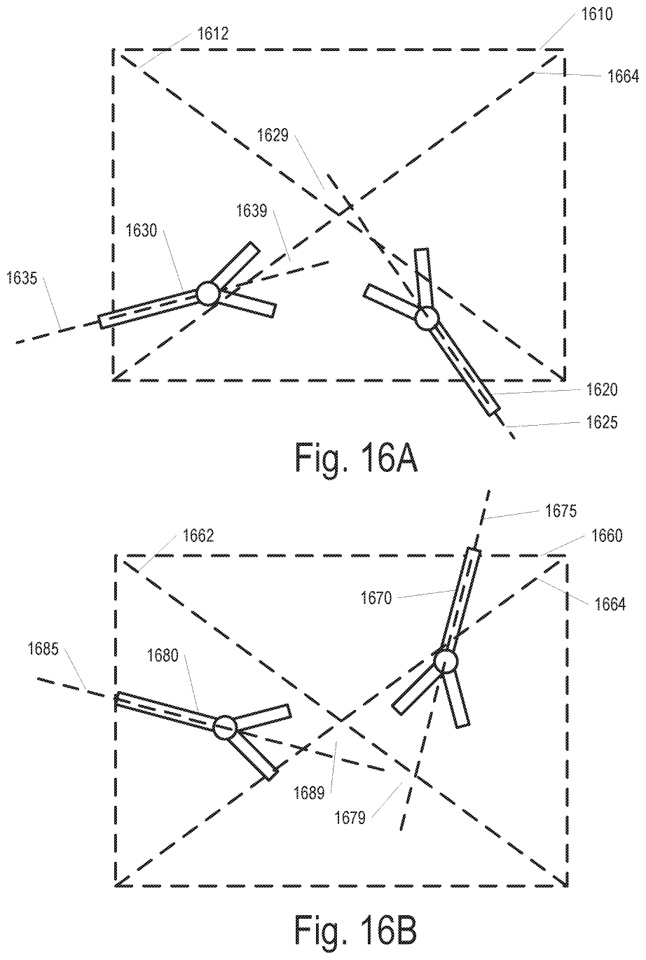

At operation 1556, an anthropomorphic metric for the port location is determined. The anthropomorphic metric captures the ease with which the operator may manipulate the end effector to the target tissue and manipulate the end effector around the target tissue using the port location. In some examples, when the surgical instrument and the end effector are to be operated so that motion of an input control device relative to a display device results in corresponding motion of the surgical instrument and end effector (e.g., the surgical instrument and end effector move as if they are a surgical instrument held in the operator's hand), the most natural approach toward the workspace may be to bring the end effector toward the target tissue from the lower left (as if held in the left hand) or from the lower right (as if held in the right hand). These concepts are shown in FIGS. 16A-16B, which are simplified diagrams of different end effector positions and orientations within a workspace according to some embodiments. FIG. 16A shows a view 1610 of a workspace that may be captured by the imaging device whose placement was determined during operation 1540 and whose end effectors are introduced into the workspace using a first set of port locations. In some examples, view 1610 may be obtained by placing the imaging device at a known imaging distance from the target tissue, which is placed at the center of view 1610. Two planes (shown as projected lines in FIG. 16A) indicate the main diagonals 1612 and 1614 of view 1610 and may roughly correspond to the ideal approach directions for surgical instruments and/or end effectors. Also shown in FIG. 16A is a first end effector 1620 that approaches a center point of the workspace along an insertion axis 1625. A difference between insertion axis 1625 and main diagonal 1612 of view 1610 is shown as angle 1629. FIG. 16A also shows a second end effector 1630 that approaches a center point of the workspace along an insertion axis 1635. A difference between insertion axis 1635 and main diagonal 1614 of view 1610 is shown as angle 1639.

As another example, FIG. 16B shows another view 1660 of a workspace that may be captured by the imaging device whose placement was determined during operation 1540 and whose end effectors are introduced into the workspace using a second set of port locations. In some examples, view 1660 may be obtained by placing the imaging device at a known imaging distance from the target tissue, which is placed at the center of view 1660. Two planes (shown as projected lines in FIG. 16B) indicate the main diagonals 1662 and 1664 of view 1660 and may roughly correspond to the ideal approach directions for surgical instruments and/or end effectors. Also shown in FIG. 16B is a first end effector 1670 that approaches a center point of the workspace along an insertion axis 1675. A difference between insertion axis 1675 and main diagonal 1662 of view 1660 is shown as angle 1679. FIG. 16B also shows a second end effector 1680 that approaches a center point of the workspace along an insertion axis 1685. A difference between insertion axis 1685 and main diagonal 1664 of view 1660 is shown as angle 1689.

Because angles 1629 and 1639 are smaller than angles 1679 and 1689, they indicate that end effectors 1620 and 1630 are approaching the center point of the workspace more naturally than end effectors 1670 and 1680. Thus, the first set of port locations, which are associated with end effectors 1620 and 1630 are considered more anthropomorphic and are assigned a higher anthropomorphic metric than the second set of port locations. In some examples, the anthropomorphic metric for a port location may be determined using either Equation 15 or Equation 16, where b corresponds to the angle between the insertion axis of the end effector from the port location and the main diagonal.

Anthropomorphic Metric=(90−b)/90 Equation 15

Anthropomorphic Metric=(180−b)/180 Equation 16

In some examples, the additional information obtained regarding the imaging device during operation 1540 (e.g., the imaging device type, the aspect ratio, the field of view, the working distance, and/or the like) may be used to help position views 1610 and/or 1660 as well as to determine the orientations of the main diagonals 1612, 1614, 1662, and/or 1664.

In some embodiments, the anthropomorphic metric may also account for a human factors constraint, such as a handedness preference of the operator. In some examples, when the operator indicates a preference for a particular surgical instrument to be used in a specific hand then the angle used for the anthropomorphic metric should be determined using the main diagonal for that hand (e.g., main diagonal 1612 and/or 1662 for a right-handed surgical instrument and main diagonal 1614 and/or 1664 for a left handed surgical instrument) even though the other main diagonal may have a smaller angle relative to the insertion axis of the surgical instrument. In some examples, both right- and left-handed anthropomorphic metrics may be determined for the port location so that both right- and left-handed evaluations may be considered during the remainder of method 1500.

Referring back to FIG. 15, at operation 1560, each of the possible combinations of port locations identified during operation 1520 is iterated through to evaluate the suitability of the combination of port locations for a procedure. When the procedure is to be performed using two surgical instruments then each combination of port locations includes two port locations. More generally, when the procedure is to be performed using n surgical instruments then each combination of port locations includes n port locations. As each of the possible combinations of port locations is considered, the analyses of operations 1562 and 1564 are repeated to determine aggregate scoring metrics usable to characterize the suitability of the combination of port locations for use with the contemplated procedure.

At operation 1562, a collision metric is determined for the combination of port locations. The collision metric is a kinematic measure providing an indication of how likely or unlikely collisions are to occur in the portions of the surgical instruments, manipulators, and/or repositionable arms located proximal to the port locations in the combination. In some examples, the collision metric may be determined based on an amount of overlap between the collision volumes determined during operation 1554 for each of the port locations in the combination. Where more overlap in the collision volumes occurs, the likelihood of a collision increases and the collision metric decreases. In some examples, the collision metric may be determined based on a percentage of overlap of each of the collision volumes by other collision volumes. In some examples, the percentage of overlap of a collision volume by other collision volumes is determined based on the ratio of the total collision volume that is overlapped by other collision volumes and the total collision volume. In some examples, this may be converted to an overlap metric as shown in Equation 5.

Overlap Metric=1−(overlapped CV)/(total CV) Equation 5

When the combination of port locations includes two port locations, the overlap metric may be used as the collision metric. When the combination of port locations includes three or more port locations, the collision metric may be determined by using an aggregation of the overlap metrics for each of the corresponding collision volumes. In some examples, the overlap metrics for each of the corresponding collision volumes may be aggregated using any triangular norm function, such as minimum, multiplication, and/or the like.

At operation 1564, an aggregate scoring metric is determined for the combination of port locations. In some examples, the aggregate scoring metric may be determined by aggregating together the reachability metric for each of the port locations in the combination, the anthropomorphic metric for each of the port locations in the combination, and the collision metric for the combination. In some examples, the aggregation may be performed using a weighted sum with the weights being pre-assigned and/or adjustable by an operator. In some examples, a weight of zero may be used to omit a corresponding metric from the aggregation. In some examples, the aggregation may be determined by combining the metrics using any triangular norm function, such as minimum, multiplication, and/or the like. In some examples, the aggregate scoring metric may be used to indicate the suitability of the combination of port locations relative to other combinations of port locations.

At operation 1570, one or more of the combinations of port locations are displayed to an operator. For example, system 100 may direct a display device to display a graphical representation of one or more of the combinations of port locations.

In some examples, a combination of port locations and a corresponding evaluation may be displayed to the operator using any suitable display device include a tablet, a computer screen, a simulator, and/or the like. In some examples, the combination of port locations and the corresponding evaluation may be displayed as a two-dimensional projection, a three-dimensional image on a stereoscopic display and/or the like. In some examples, the order in which the combinations of port locations may be displayed may be based on their relative aggregate scoring metrics, with the highest scoring combination being displayed first. In some examples, one or more lists, menus, and/or the like may be used to allow the operator to select from among the evaluated combinations. In some examples, the corresponding evaluation may be displayed as one or more text lines indicating the values determined for each of the reachability, anthropomorphic, and/or collision metrics along with the aggregate scoring metric. In some examples, the one or more text lines may indicate the relative weighting of each metric and optionally provide mechanisms for the operator to adjust the weights. In some examples, one or more mechanisms for adding additional constraints (e.g., human factors constraints such as handedness of one of the surgical instruments) may also be provided.

At operation 1580, port location selections are received from the operator. In some examples, the port location selections may be selected by indicating that a current combination of port locations being displayed (e.g., using process 1570) is the selected combination. In some examples, other selection mechanisms may be used, such as selecting from a list, and/or the like.

At process 1590, guidance is provided to the operator for the placing of ports at the port locations selected during process 1580. In some examples, the guidance for the placing of a port at one of the selected port locations may include one or more of laser targets projected on the port locations, pointing to the port location using the manipulator, projections onto the patient, haptic guidance for manual positioning of the manipulator, augmented reality overlays on a stereoscopic image of the patient, and/or the like.

In some examples, system 100 may perform operation 202 by identifying, based on external body wall data 206 and internal depth data 208, a set-up position for a manipulator arm of computer-assisted surgical system 204. System 100 may then instruct computer-assisted surgical system 204 to configure the manipulator arm in the set-up position. These operations may be performed in any suitable manner. For example, the set-up position may be selected such that the manipulator arm does not come in contact with the patient and/or another manipulator arm while a surgical instrument connected to the manipulator arm is being inserted into the patient and/or while the surgical instrument is being used within the patient. In some examples, the set-up position may be further determined based on kinematics data generated by computer-assisted surgical system 204. In some examples, the set-up position is determined by determining a position of one or more set-up joints of the manipulator arm.

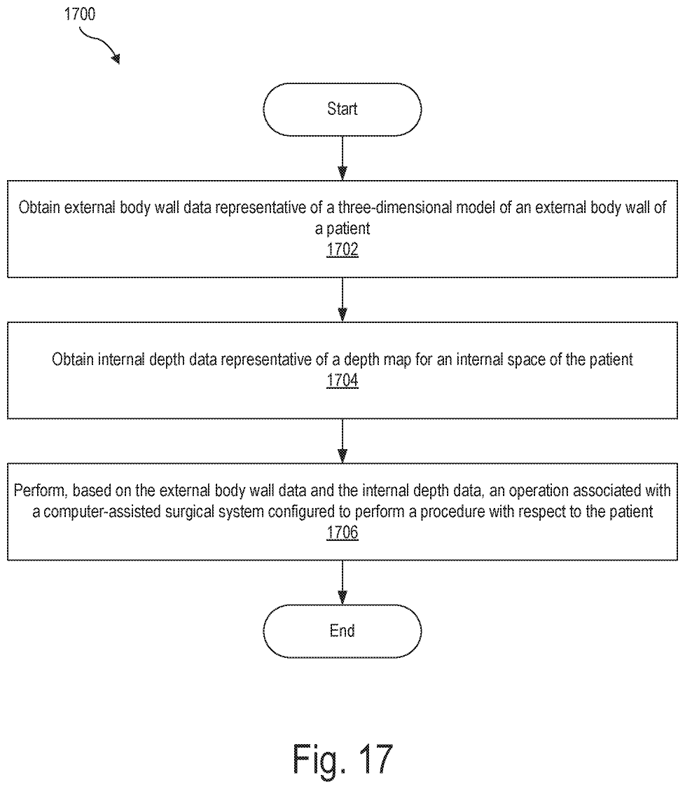

FIG. 17 illustrates an exemplary method 1700 that may be performed by an operation management system (e.g., system 100 and/or any implementation thereof). While FIG. 17 illustrates exemplary operations according to one embodiment, other embodiments may omit, add to, reorder, and/or modify any of the operations shown in FIG. 17.

In operation 1702, an operation management system obtains external body wall data representative of a three-dimensional model of an external body wall of a patient. Operation 1702 may be performed in any of the ways described herein.

In operation 1704, the operation management system obtains internal depth data representative of a depth map for an internal space of the patient. Operation 1704 may be performed in any of the ways described herein.

In operation 1706, the operation management system performs, based on the external body wall data and the internal depth data, an operation associated with a computer-assisted surgical system configured to perform a procedure with respect to the patient. Operation 1706 may be performed in any of the ways described herein.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g. a hard disk, a floppy disk, magnetic tape, etc.), ferroelectric random-access memory (“RAM”), and an optical disc (e.g., a compact disc, a digital video disc, a Blu-ray disc, etc.). Exemplary volatile storage media include, but are not limited to, RAM (e.g., dynamic RAM).

FIG. 17 illustrates an exemplary computing device 1700 that may be specifically configured to perform one or more of the processes described herein. Any of the systems, computing devices, and/or other components described herein may be implemented by computing device 1700.

As shown in FIG. 17, computing device 1700 may include a communication interface 1702, a processor 1704, a storage device 1706, and an input/output (“I/O”) module 1708 communicatively connected one to another via a communication infrastructure 1710. While an exemplary computing device 1700 is shown in FIG. 17, the components illustrated in FIG. 17 are not intended to be limiting. Additional or alternative components may be used in other embodiments. Components of computing device 1700 shown in FIG. 17 will now be described in additional detail.

Communication interface 1702 may be configured to communicate with one or more computing devices. Examples of communication interface 1702 include, without limitation, a wired network interface (such as a network interface card), a wireless network interface (such as a wireless network interface card), a modem, an audio/video connection, and any other suitable interface.

Processor 1704 generally represents any type or form of processing unit capable of processing data and/or interpreting, executing, and/or directing execution of one or more of the instructions, processes, and/or operations described herein. Processor 1704 may perform operations by executing computer-executable instructions 1712 (e.g., an application, software, code, and/or other executable data instance) stored in storage device 1706.

Storage device 1706 may include one or more data storage media, devices, or configurations and may employ any type, form, and combination of data storage media and/or device. For example, storage device 1706 may include, but is not limited to, any combination of the non-volatile media and/or volatile media described herein. Electronic data, including data described herein, may be temporarily and/or permanently stored in storage device 1706. For example, data representative of computer-executable instructions 1712 configured to direct processor 1704 to perform any of the operations described herein may be stored within storage device 1706. In some examples, data may be arranged in one or more databases residing within storage device 1706.

I/O module 1708 may include one or more I/O modules configured to receive user input and provide user output. I/O module 1708 may include any hardware, firmware, software, or combination thereof supportive of input and output capabilities. For example, I/O module 1708 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1708 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1708 is configured to provide graphical data to a display for presentation to a user. The graphical data may be representative of one or more graphical user interfaces and/or any other graphical content as may serve a particular implementation.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A system comprising:
a memory storing instructions; and
a processor communicatively coupled to the memory and configured to execute the instructions to:
obtain external body wall data representative of a three-dimensional model of an external body wall of a patient and based on external depth data for the external body wall;
obtain internal depth data representative of a depth map for an internal space of the patient; and
perform, based on the external body wall data and the internal depth data, an operation associated with a computer-assisted surgical system configured to perform a procedure with respect to the patient.

2. The system of claim 1, wherein the obtaining of the internal depth data comprises directing a depth sensor in an imaging device to acquire the internal depth data while the depth sensor is aimed at the internal space through a camera port formed through the external body wall of the patient.

3. The system of claim 2, wherein the obtaining of the external body wall data comprises:
directing the depth sensor in the imaging device to acquire the external depth data by scanning the external body wall while the imaging device is external to the patient;
receiving the external depth data from the depth sensor; and
using the external depth data as the external body wall data representative of the three-dimensional model of the external body wall of the patient.

4. The system of claim 3, wherein:
the imaging device is attached to a manipulator arm of the computer-assisted surgical system while the depth sensor acquires the external depth data and the internal depth data;
the computer-assisted surgical system is configured to generate kinematics data for the imaging device while the depth sensor acquires the external depth data and the internal depth data;
the processor is further configured to execute the instructions to register, based on the kinematics data, the external body wall data with the internal depth data; and
the performance of the operation is based on the registration of the external body wall data with the internal depth data.

5. The system of claim 2, wherein the imaging device further comprises a visible light camera configured to acquire a visible light image of the internal space.

6. The system of claim 1, wherein the obtaining of the external body wall data comprises:
directing a first visible light camera included in an imaging device to acquire a first image of the external body wall;
directing a second visible light camera included in the imaging device to acquire a second image of the external body wall; and
generating, based on the first and second images, the external depth data.

7. The system of claim 1, wherein the obtaining of the external body wall data comprises scanning the external body wall of the patient using at least one of a computer-aided tomography (CT) scanner, a magnetic resonance imaging (MRI) device, an ultrasound device, or a three-dimensional scanning (LIDAR) device.

8. The system of claim 1, wherein the performing of the operation comprises identifying, based on the external body wall data and the internal depth data, a port location on the external body wall of the patient through which the computer-assisted surgical system is to insert a surgical instrument into the internal space of the patient.

9. The system of claim 8, wherein the identifying of the port location comprises using the external body wall data and the internal depth data to identify a positioning on the external body wall for the port location that allows the surgical instrument to access, through the port location, a structure within the internal space while avoiding collision with an additional surgical instrument.

10. The system of claim 8, wherein the identifying of the port location is further based on kinematics data generated by the computer-assisted surgical system.

11. The system of claim 8, wherein the surgical instrument is attached to a manipulator arm of the computer-assisted surgical system.

12. The system of claim 11, wherein the identifying of the port location comprises identifying the port location such that the surgical instrument is configured to access a structure within the internal space without the manipulator arm colliding with a different manipulator arm.

13. The system of claim 11, wherein the identifying the port location comprises identifying the port location such that the surgical instrument and the manipulator arm avoid unintentional contact with the patient.

14. The system of claim 8, wherein the processor is further configured to execute the instructions to direct a display device to display a graphical representation of the port location.

15. The system of claim 8, wherein:
the processor is further configured to execute the instructions to determine, for a candidate port location, at least one of
a reachability metric indicating an ability of the surgical instrument to reach a target structure located in the internal space of the patient using a candidate port location,
an anthropomorphic metric indicating an ease with which a user may manipulate the surgical instrument introduced into the internal space of the patient through the candidate port location,
a collision volume for portions of the computer-assisted surgical system proximal to the candidate port location, the collision volume corresponding to a volume swept by the portions of the computer-assisted surgical system proximal to the candidate port location, or
a collision metric indicating a likelihood of a collision between portions of the computer-assisted surgical system proximal to the candidate port location; and
the identifying of the port location is further based on at least one of the reachability metric, the anthropomorphic metric, the collision volume, or the collision metric.

16. The system of claim 1, wherein the performing of the operation comprises identifying, based on the external body wall data and the internal depth data, a set-up position for a manipulator arm of the computer-assisted surgical system.

17. The system of claim 16, wherein the identifying of the set-up position is further based on kinematics data generated by the computer-assisted surgical system.

18. (canceled)

19. A method comprising:
obtaining, by an operation management system, external body wall data representative of a three-dimensional model of an external body wall of a patient and based on external depth data for the external body wall;
obtaining, by the operation management system, internal depth data representative of a depth map for an internal space of the patient; and
performing, by the operation management system based on the external body wall data and the internal depth data, an operation associated with a computer-assisted surgical system configured to perform a procedure with respect to the patient.

20-36. (canceled)

37. A non-transitory computer-readable medium storing instructions that, when executed, direct a processor of a computing device to:
obtain external body wall data representative of a three-dimensional model of an external body wall of a patient and based on external depth data for the external body wall;
obtain internal depth data representative of a depth map for an internal space of the patient; and
perform, based on the external body wall data and the internal depth data, an operation associated with a computer-assisted surgical system configured to perform a procedure with respect to the patient.

38. The system of claim 1, wherein the external depth data and the internal depth data are both generated during a surgical procedure during which the computer-assisted surgical system is configured to perform the procedure with respect to the patient.
